(19) United States
(12) Patent Application Publication
(10) Pub. No.: US 2017/0184593 A1
(43) Pub. Date: Jun. 29, 2017

(54) A chronic renal disease diagnostic kit

(71) Applicant: WENZHOU MEDICAL UNIVERSITY, Zhejiang (CN)

(72) Inventors: Xiaokun Li, Zhejiang (CN); Guang Liang, Zhejiang (CN); Xiaojie Wang, Zhejiang (CN); Shijun Li, Changchun (CN); Peisheng Hu, Covina, CA (US)

(21) Appl. No.: 15/300,236

(22) PCT Filed: Dec. 18, 2015
(86) PCT No.: PCT/CN2015/097926

§ 371 Date: Sep. 28, 2016

(30) Foreign Application Priority Data
Dec. 23, 2014 (CN) 201410811167.6

(51) Int. Cl. G01N 33/577

(52) U.S. Cl. CPC G01N 33/577; G01N 2800/347

(57) ABSTRACT

This invention publicizes a chronic renal disease diagnostic kit, comprised of FGF23 monoclonal antibody, biotin labeling reagent Sulfo-NHS-LC-Biotin, PBS buffer solution (pH7.0, 0.1M), casein saline solution, streptomycin avidin and substrate TMB. The labeling condition is the weight ratio of FGF23 antibody and Sulfo-NHS-LC-Biotin, which is 1:5-1:12. The dilution ratio of streptomycin avidin is 1:3000. The lowest testing threshold limit value of FGF23 kit is 10 pg/ml, whose sensitivity is 10 times higher than that of common monoclonal antibody, which is very significant for early diagnose of chronic renal disease.

### TECHNICAL FIELD

The invention belongs to disease detecting technical field, which specifically involves a chronic renal disease diagnostic kit.

### BACKGROUND TECHNOLOGY

Fibroblast growth factors 23 (FGF23) is a member of super-family FGFs, which plays an important role in maintaining phosphorus steady state of patient with chronic renal disease in early stage by suppressing the re-absorption and enhancing the external secretion of phosphorus (J Am Soc Nephro 1.2007:18:1637-1647). According to the reports from the American Journal of Kidney and the New England Journal, increase of FGF23 concentration occurs before abnormal phosphorus metabolism, and the expression level of FGF23 is independently related to the deterioration of chronic renal disease and the mortality of patients receiving dialysis treatment in late stage. Therefore, FGF23 becomes a significant marker of early diagnose of renal function. The currently clinically used bio-marker creatinine has its disadvantages such as low accuracy and specificity, so once it is detected that the creatinine level exceeds standard, the course of disease has already reached the middle or late stage in most cases. Hence, the development of new diagnostic reagent of chronic renal disease—FGF23 monoclonal antibody kit is of important clinical application value.

### INVENTION CONTENTS

The invention aims to provide a chronic renal disease diagnostic kit, offering an easy method for early diagnose of chronic renal disease, and provide a new approach for detection and treatment of chronic renal disease.

The invention is specifically realized by the following technical plans:

A chronic renal disease diagnostic kit is comprised of FGF23 monoclonal antibody, Sulfo-NHS-LC-Biotin, buffer solution, casein solution, streptomycin avidin and substrate TMB.

The kit of the invention uses PBS (pH7.0, 0.1M) as coating buffer solution. The coating volume is 100 μl, and the coating concentration is 2 μg/ml.

The labeling condition of the kit is the weight ratio of FGF23 monoclonal antibody and Sulfo-NHS-LC-Biotin, which is 1:5˜1:12; the concentration of mixture is 2 μg/ml.

The kit of the invention, wherein the said casein solution is the diluent comprising FGF23, detection antibody and streptomycin avidin; dilution ratio of streptomycin avidin is 1:3000

The kit of the invention, wherein the reaction time of the said streptomycin avidin and substrate is 10 minutes.

The kit of the invention, wherein the threshold limit value of FGF23 is 10 pg/ml, and the linear testing range is 2400 pg/ml˜10 pg/ml.

The beneficial effect of the invention is that it provides an external test kit for early detection of chronic renal disease; the sensitivity of the kit to clinical detection of FGF23 reaches 10 pg/ml, which is 10 times higher than that of common monoclonal antibody and has very important meaning for early diagnose of chronic renal disease.

### BRIEF DESCRIPTION OF FIGURE

FIG. 1 is the linear relationship of absorbancy value and sample concentration in the invention implementation.

### SPECIFIC IMPLEMENTATION METHODS

A further description of the invention will be done by introducing implementations. The following content is only an example of a comparatively good implementation of the invention, and there is are no other forms of limitation for the invention, so it is possible for any technician who is familiar with this specialty to can make use of the above technical contents and make some changes to achieve equivalent implementations. Any simple or similar change of the above implementation that is not separated from the invention contents or is made according to the technical essence shall be included in the protection range of this invention.

Implementation 1 Preparation of the FGF23 Test Kit

Use FGF23-Fc antigen immunized mice to acquire hybridoma positive FGF23 antibody cloning. Three antibodies numbered as 278, 6B12 and 6H1 are selected from 1276 clones by testing their binding specificity with FGF23 protein.

The three filtered FGF23 monoclonal antibody 278, 6B12 and 6H1 are used to optimize the testing condition of FGF23 ELISA test kit.

1) Take 6B12 and 6H1 (2 mg/ml) 100 μl respectively, and proceed biotinlization reaction according the ratio of 1:20, 1:10 and 1:5 for 30 minutes at room temperature; use 1% BSA of equivalent volume to end the reaction when reaction is over; take antibody 278 for coating; dilute FGF 23 (3 ng/ml) into three times gradient diluent; test the biotinylation antibody and observe the biotinylation efficiency. It should choose NHS-PEG4-Biotin (Lot#ND172078) as the biotin reagent. (see details in Table 1)

TABLE 1
Biotin Labeling Ratio Optimization

| 6B12 | | | | 6H1 | | | |
|---|---|---|---|---|---|---|---|
| 30 | 20 | 10 | 5 | 30 | 20 | 10 | 5 |
| 1.554 | 1.517 | 3.11 | 3.091 | 0.911 | 1.212 | 3.761 | 2.462 |
| 0.507 | 0.698 | 0.767 | 1.179 | 0.418 | 0.489 | 1.85 | 0.879 |
| 0.204 | 0.367 | 0.334 | 0.538 | 0.232 | 0.269 | 0.813 | 0.433 |
| 0.141 | 0.181 | 0.198 | 0.258 | 0.146 | 0.147 | 0.306 | 0.187 |
| 0.107 | 0.137 | 0.109 | 0.137 | 0.103 | 0.116 | 0.219 | 0.145 |
| 0.084 | 0.076 | 0.093 | 0.09 | 0.098 | 0.092 | 0.13 | 0.114 |
| 0.139 | 0.078 | 0.077 | 0.09 | 0.112 | 0.106 | 0.109 | 0.107 |

It can be seen from Table 1 that the best ratio for biotin labeling of antibody 6B12 is 1:15 and the optimal concentration for biotin labeling of 6H1 is 1:10.

Based on the above experiment results, optimization of the bio-labeling was further carried out around the optimal biotinylation condition. Biotinylation reactions were done according to 1:8, 1:6, 1:5 and 1:4 for 6B12, and 1:12, 1:10 and 1:8 for 6H1.

TABLE 2
Biotin Labeling Ratio Optimization

| | |
|---|---|
| 6B12 | 6H1 |

| 8 | 6 | 5 | 4 | 12 | 10 | 8 |
|---|---|---|---|---|---|---|
| 1.035 | 0.314 | 0.5 | 0.383 | 0.347 | 0.572 | 0.472 |
| 0.589 | 0.158 | 0.291 | 0.227 | 0.182 | 0.401 | 0.459 |
| 0.381 | 0.108 | 0.233 | 0.174 | 0.144 | 0.299 | 0.262 |
| 0.271 | 0.09 | 0.184 | 0.121 | 0.126 | 0.264 | 0.23 |
| 0.256 | 0.086 | 0.177 | 0.138 | 0.136 | 0.28 | 0.195 |
| 0.262 | 0.082 | 0.185 | 0.139 | 0.12 | 0.247 | 0.207 |
| 0.252 | 0.079 | 0.158 | 0.131 | 0.105 | 0.231 | 0.212 |
| 0.241 | 0.082 | 0.189 | 0.135 | 0.132 | 0.257 | 0.226 |

Research data in Table 2 revealed that the best ratio for biotin labeling of antibody 6B12 was 1:8 and the optimal concentration for biotin labeling of 6H1 was 1:10.

Based on the above experiment results, optimization of the bio-labeling was further carried out around the optimal biotinylation condition. Biotinylation reactions were done according to 1:7, 1:8 and 1:9 for 6B12, and 1:9, 1:10 and 1:11 for 6H1.

It was showed in Table 3, the best ratio for biotin labeling of antibody 6B12 was 1:8 and the optimal concentration for biotin labeling of 6H1 was 1:10 and 1:9.

TABLE 3
Biotin Labeling Ratio Optimization

| 6H1 | | | 6B12 | | |
|---|---|---|---|---|---|
| 9 | 10 | 11 | 7 | 8 | 9 |
| 3.969 | 3.792 | 2.966 | 2.865 | 3.461 | 2.47 |
| 2.257 | 2.624 | 2.142 | 1.255 | 1.796 | 1.475 |
| 1.595 | 1.47 | 1.152 | 0.872 | 0.979 | 0.747 |
| 0.834 | 0.726 | 0.481 | 0.368 | 0.455 | 0.307 |
| 0.296 | 0.289 | 0.192 | 0.179 | 0.2 | 0.157 |
| 0.181 | 0.161 | 0.137 | 0.119 | 0.12 | 0.103 |
| 0.099 | 0.095 | 0.086 | 0.075 | 0.077 | 0.072 |
| 0.074 | 0.069 | 0.063 | 0.061 | 0.06 | 0.057 |

2) The labeling sensitivity differences of Sulfo-NHS-LC-Biotin (Lot#21327) and NHS-PEG4-Biotin (Lot#ND172078) from Thermo scientific company were studied. See results in Table 4 and Table 5. According to the experiment data and a comprehensive consideration of sensitivity and background level, Sulfo-NHS-LC-Biotin (Lot#21327) was selected as the final biotin labeling reagent.

TABLE 4
Labeling data of Sulfo-NHS-LC-Biotin

| 6B12 | | | | 6H1 | | | |
|---|---|---|---|---|---|---|---|
| 30 | 20 | 10 | 5 | 30 | 20 | 10 | 5 |
| 3.389 | 3.618 | 3.511 | 2.582 | 0.786 | 1.541 | 2.645 | 2.937 |
| 3.269 | 1.792 | 2.865 | 2.255 | 1.286 | 1.531 | 1.83 | 3.752 |
| 1.893 | 2.058 | 2.364 | 1.665 | 0.859 | 1.047 | 2.371 | 2.891 |
| 0.738 | 0.911 | 1.023 | 0.772 | 0.376 | 10.482 | 1 | 0.937 |
| 0.305 | 0.367 | 0.486 | 0.363 | 0.272 | 0.255 | 0.45 | 0.525 |
| 0.208 | 0.254 | 0.238 | 0.19 | 0.113 | 0.196 | 0.226 | 0.243 |
| 0.147 | 0.183 | 0.218 | 0.145 | 0.123 | 0.131 | 0.151 | 0.174 |
| 0.096 | 0.076 | 0.085 | 0.094 | 0.103 | 0.08 | 0.079 | 0.086 |

TABLE 5
Labeling data of NHS-PEG4-Biotin

| 6B12 | | | | 6H1 | | | |
|---|---|---|---|---|---|---|---|
| 30 | 20 | 10 | 5 | 30 | 20 | 10 | 5 |
| 1 | 2 | 3 | 4 | 5 | 6 | 7 | 8 |
| 2.27 | 2.548 | 2.73 | 1.932 | 1.993 | 2.449 | 2.697 | 1.591 |
| 2.064 | 2.335 | 2.366 | 1.735 | 2.071 | 2.441 | 2.67 | 1.795 |
| 1.701 | 1.658 | 1.555 | 0.965 | 1.342 | 1.733 | 1.711 | 1.153 |
| 0.655 | 0.838 | 0.902 | 0.532 | 0.601 | 0.732 | 0.861 | 0.609 |
| 0.28 | 0.352 | 0.427 | 0.305 | 0.315 | 0.447 | 0.447 | 0.378 |
| 0.145 | 0.179 | 0.204 | 0.159 | 0.229 | 0.215 | 0.264 | 0.173 |
| 0.131 | 0.141 | 0.153 | 0.138 | 0.144 | 0.149 | 0.169 | 0.128 |
| 0.091 | 0.085 | 0.118 | 0.115 | 0.095 | 0.098 | 0.119 | 0.106 |

3) According to data in Table 4, optimization of labeling condition and refinement of labeling ratio of Sulfo-NHS-LC-Biotin (Lot#21327) were further carried out around the best sensitivity labeling data of 6B12 and 6H1. Biotinylation reactions were done according to 1:8, 1:9, 1:10, 1:11, 1:12 and 1:20 for 6B12, and 1:3, 1:4, 1:5, 1:6, 1:7 and 1:10 for 6H1 and the results were recorded in Table 6. Based on data in Table 4, refinement of labeling ratio was done around the best sensitivity data of 6B12 and 6H1. Biotinylation reactions were done according to 1:8, 1:9, 1:10, 1:11 and 1:12 for 6B12 and 6H1 respectively, and the experiment data were recorded in Table 7. Experiment data of Table 7 showed that the test sensitivity were highest when labeling ratio was 1:8 for 6B12, and 1:10 for 6H1.

TABLE 6
Biotin Labeling Ratio Optimization of 6B12 and 6H1

| 6B12 | | | | | | 6H1 | | | | | |
|---|---|---|---|---|---|---|---|---|---|---|---|
| 8 | 9 | 10 | 11 | 12 | 20 | 10 | 7 | 6 | 5 | 4 | 3 |
| 2.868 | 1.95 | 2.032 | 1.58 | 1.71 | 1.626 | 2.801 | 2.303 | 2.579 | 1.981 | 1.861 | 0.86 |
| 1.132 | 0.82 | 0.756 | 0.656 | 0.651 | 0.547 | 1.131 | 0.926 | 0.864 | 0.799 | 0.737 | 0.437 |
| 0.378 | 0.304 | 0.356 | 0.254 | 0.28 | 0.253 | 0.472 | 0.426 | 0.414 | 0.293 | 0.35 | 0.223 |
| 0.189 | 0.164 | 0.192 | 0.132 | 0.165 | 0.167 | 0.286 | 0.19 | 0.179 | 0.178 | 0.171 | 0.132 |
| 0.147 | 0.134 | 0.124 | 0.144 | 0.155 | 0.176 | 0.153 | 0.145 | 0.133 | 0.116 | 0.166 | 0.098 |
| 0.07 | 0.09 | 0.091 | 0.072 | 0.126 | 0.142 | 0.082 | 0.078 | 0.086 | 0.087 | 0.101 | 0.078 |

TABLE 7
Biotin Labeling Ratio Optimization of 6B12 and 6H1

| 6B12 | | | | | 6H1 | | | | |
|---|---|---|---|---|---|---|---|---|---|
| 8 | 9 | 10 | 11 | 12 | 8 | 9 | 10 | 11 | 12 |
| 2.87 | 2.53 | 2.516 | 2.475 | 2.234 | 2.413 | 2.403 | 2.769 | 2.45 | 2.335 |
| 1.056 | 0.98 | 1.029 | 0.814 | 0.874 | 1.08 | 1.016 | 1.268 | 1.311 | 1.116 |
| 0.514 | 0.344 | 0.383 | 0.425 | 0.358 | 0.463 | 0.418 | 0.589 | 0.501 | 0.469 |
| 0.258 | 0.183 | 0.181 | 0.229 | 0.183 | 0.232 | 0.226 | 0.251 | 0.237 | 0.206 |
| 0.155 | 0.17 | 0.127 | 0.153 | 0.13 | 0.154 | 0.138 | 0.169 | 0.146 | 0.187 |
| 0.094 | 0.106 | 0.094 | 0.093 | 0.098 | 0.09 | 0.093 | 0.104 | 0.095 | 0.091 |

4) Selection of the best testing concentration was done for 6B12, 6H1 and mixture of the two at 1 μg/m and 2 μg/ml respectively. Experiment data were seen in Table 8.

TABLE 8
Selection of the best testing concentration of antibody

| | | |
|---|---|---|
| 6B12 | 6H1 | 6B12 + 6H1 |

| 2 μg/ml | 1 μg/ml | 2 μg/ml | 1 μg/ml | 2 μg/ml | 1 μg/ml |
|---|---|---|---|---|---|
| 1.278 | 0.568 | 1.416 | 1.639 | 1.595 | 1.362 |
| 0.508 | 0.262 | 0.61 | 0.544 | 0.76 | 0.615 |
| 0.219 | 0.138 | 0.309 | 0.197 | 0.354 | 0.302 |
| 0.13 | 0.114 | 0.156 | 0.109 | 0.18 | 0.152 |
| 0.09 | 0.078 | 0.106 | 0.103 | 0.113 | 0.111 |
| 0.102 | 0.109 | 0.092 | 0.085 | 0.097 | 0.081 |
| 0.08 | 0.067 | 0.094 | 0.068 | 0.091 | 0.082 |
| 0.072 | 0.071 | 0.085 | 0.068 | 0.088 | 0.09 |

5) Selection of coating buffer solution

Use phosphate buffer solution (0.1M, pH7.0) and sodium borohydride solution (0.1M, pH9.0) as coating solutions and then test the detection sensitivity and background level respectively. The experiment data were recorded in Chart 9. According to the data in Chart 9, after a comprehensive consideration of detection sensitivity and background level, phosphate buffer solution (pH7.0) was selected as the coating solution of FGF23 kit.

TABLE 9
Comparison of coating efficiency of phosphate
and sodium borohydride solution

| | | |
|---|---|---|
| | Sodiun | Sodium |
| PBS | tetraborate | tetraborate |

| | | | | | |
|---|---|---|---|---|---|
| 2.095 | 2 | 1.715 | 1.763 | 1.809 | 1.897 |
| 0.704 | 0.712 | 0.718 | 0.73 | 0.67 | 0.672 |
| 0.341 | 0.33 | 0.329 | 0.344 | 0.311 | 0.306 |
| 0.199 | 0.215 | 0.22 | 0.209 | 0.171 | 0.175 |
| 0.151 | 0.161 | 0.169 | 0.18 | 0.137 | 0.129 |
| 0.136 | 0.144 | 0.183 | 0.152 | 0.121 | 0.11 |
| 0.142 | 0.126 | 0.165 | 0.146 | 0.113 | 0.105 |
| 0.124 | 0.118 | 0.146 | 0.126 | 0.112 | 0.106 |

6) Influence of coating volume on detection sensitivity and background level

Use different test antibodies of different labeling ratios to test the detection sensitivity and background level of anti-FGF23 at 100p1 and 200p1 coating volumes, and select the optimal coating volume. Experiment data were recorded in Table 10. According to data in Table 10, increase of coating volume leads to a better detection sensitivity, but it also raises the background level. Compared with sensitivity, background level had a greater influence on detection of clinical samples. Therefore, 100 μl coating volume was chosen to be used as anti-FGF23 coating volume through a comprehensive consideration.

TABLE 10
Influence of different coating volume on the detection
sensitivity and background level of FGF23 kit

| 6B12 | | | | | | 6H1 | | | | | |
|---|---|---|---|---|---|---|---|---|---|---|---|
| 100 μl | | | | | | 200 μl | | | | | |
| 7 | 8 | 9 | 9 | 10 | 11 | 7 | 8 | 9 | 9 | 10 | 11 |
| 1.221 | 1.253 | 1.112 | 0.889 | 1.13 | 0.997 | 2.301 | 2.659 | 2.324 | 1.914 | 2.382 | 1.773 |
| 0.541 | 0.573 | 0.457 | 0.404 | 0.487 | 0.384 | 0.96 | 1.091 | 0.923 | 0.794 | 0.942 | 0.741 |
| 0.27 | 0.23 | 0.212 | 0.197 | 0.216 | 0.186 | 0.418 | 0.458 | 0.383 | 0.359 | 0.436 | 0.31 |
| 0.146 | 0.153 | 0.115 | 0.116 | 0.12 | 0.114 | 0.239 | 0.256 | 0.213 | 0.199 | 0.231 | 0.182 |
| 0.097 | 0.116 | 0.088 | 0.084 | 0.087 | 0.078 | 0.162 | 0.169 | 0.145 | 0.133 | 0.149 | 0.13 |
| 0.063 | 0.07 | 0.061 | 0.058 | 0.07 | 0.067 | 0.106 | 0.112 | 0.096 | 0.091 | 0.096 | 0.093 |

7) Influence of anti-FGF23 coating concentration on detection sensitivity and background level

Take 2 μg/ml, 4 μg/ml, 8 μg/ml, 16 μg/ml, 20 μg/ml as anti-FGF23 coating concentrations to detect the influence of different coating concentration on detection sensitivity and background level of FGF23 test kit. Results were seen in Table 11.

TABLE 11
Influence of different coating concentration of anti-FGF23on
detection sensitivity and background level of FGF23 test kit

| 2 μg/ml | | 4 μg/ml | | 8 μg/ml | | 16 μg/ml | | 20 μg/ml | |
|---|---|---|---|---|---|---|---|---|---|
| 1.955 | 1.832 | 1.836 | 1.809 | 1.912 | 1.823 | 1.906 | 1.809 | 1.998 | 1.966 |
| 0.632 | 0.68 | 0.661 | 0.605 | 0.67 | 0.688 | 0.677 | 0.695 | 0.744 | 0.718 |
| 0.302 | 0.294 | 0.292 | 0.261 | 0.294 | 0.01 | 0.305 | 0.317 | 0.324 | 0.329 |
| 0.177 | 0.173 | 0.171 | 0.167 | 0.181 | 0.171 | 0.158 | 0.172 | 0.155 | 0.165 |
| 0.122 | 0.116 | 0.157 | 0.141 | 0.126 | 0.12 | 0.124 | 0.138 | 0.144 | 0.127 |
| 0.073 | 0.08 | 0.095 | 0.096 | 0.11 | 0.087 | 0.105 | 0.106 | 0.099 | 0.087 |

Data in Table 11 showed that when anti-FGF23 coating concentration increased, there was no significant improvement of detection sensitivity, but there was some corresponding increase of background level. Therefore, anti-FGF23 coating concentration should be of 2 μg/ml.

8) Influence of enzyme concentration and reaction time of enzyme and substrate on detection sensitivity

Use 1:2000 and 1:3000 streptomycin avidin diluents and set 5 and 10 minutes as the reaction time of streptomycin avidin and TMB to evaluate the influence of enzyme concentration and reaction time of enzyme and substrate on detection sensitivity and background level. Experiment data were seen in Table 12. According to Table 12, the influence of different concentrations (1:2000 and 1:3000 streptomycin avidin dilution) on detection sensitivity of FGF23 was not significant. When the reaction time was 10 minutes, it can improve FGF23 detection sensitivity and meanwhile the increase of background level was not significant. Therefore, 1:3000 was selected as enzyme reaction concentrate, and 10 minutes as enzyme reaction time.

TABLE 12
Influence of streptomycin avidin dilution and the reaction time of enzyme and substrate on detection sensitivity

| 6H1 | | | | 6B12 | | | | [symbol] 1 + 6B12 | | | |
|---|---|---|---|---|---|---|---|---|---|---|---|
| 5 MIN | 10 MIN | 5 MIN | 10 MIN | 5 MIN | 10 MIN | 5 MIN | 10 MIN | 5 MIN | 10 MIN | 5 MIN | 101 MIN |
| 1.19 | 1.495 | 1.167 | 1.561 | 2.239 | 2.913 | 2.21 | 2.838 | 2.585 | 3.259 | 2.44 | 3.069 |
| 0.449 | 0.576 | 0.423 | 0.576 | 0.812 | 1.065 | 0.812 | 1.008 | 1.058 | 1.23 | 0.902 | 1.254 |
| 0.21 | 0.24 | 0.192 | 0.253 | 0.331 | 0.421 | 0.352 | 0.401 | 0.523 | 0.46 | 0.379 | 0.531 |
| 0.114 | 0.141 | 0.114 | 0.139 | 0.174 | 0.202 | 0.165 | 0.211 | 0.226 | 0.268 | 0.194 | 0.266 |
| 0.089 | 0.102 | 0.082 | 0.111 | 0.122 | 0.125 | 0.113 | 0.134 | 0.133 | 0.156 | 0.121 | 0.178 |
| 0.082 | 0.085 | 0.093 | 0.078 | 0.102 | 0.107 | 0.093 | 0.107 | 0.102 | 0.129 | 0.102 | 0.152 |
| 0.096 | 0.079 | 0.079 | 0.087 | 0.096 | 0.094 | 0.091 | 0.095 | 0.096 | 0.113 | 0.091 | 0.114 |
| 0.068 | 0.075 | 0.065 | 0.071 | 0.074 | 0.088 | 0.076 | 0.088 | 0.091 | 0.099 | 0.087 | 0.109 |
| 2000 | 2000 | 3000 | 3000 | 2000 | 2000 | 3000 | 3000 | 2000 | 2000 | 3000 | 3000 |

9) Influence of sample diluent on background level

Use mixture of casein, 0.1MPBS and 0.05% tween 20, and mixture of 1% BSA and 0.05% tween as the diluent of FGF23, test antibody and enzyme to study the influence of sample diluent on background level. Experiment data were recorded in Table 13.

TABLE 13
Influence of different sample diluent on background level

| | | |
|---|---|---|
| Casein | Pbs + 0.05% | 1% BSA + |
| Sodium | tween | 0.05% tveen |

| | | | | | |
|---|---|---|---|---|---|
| 1.192 | 0.951 | 1.141 | 0.964 | 1.202 | 1.109 |
| 0.631 | 0.494 | 0.577 | 0.512 | 0.524 | 0.663 |
| 0.26 | 0.26 | 0.256 | 0.262 | 0.278 | 0.342 |
| 0.124 | 0.121 | 0.159 | 0.155 | 0.186 | 0.218 |
| 0.102 | 0.093 | 0.13 | 0.13 | 0.159 | 0.161 |
| 0.088 | 0.085 | 0.109 | 0.118 | 0.151 | 0.153 |
| 0.097 | 0.085 | 0.117 | 0.114 | 0.132 | 0.143 |
| 0.087 | 0.089 | 0.108 | 0.112 | 0.133 | 0.145 |

According to Table 13, the background level was at the lowest point when using casein sample diluent. A further study concerning this part will be done in future experiment.

In conclusion, this reagent kit uses Sulfo-NHS-LC-Biotin (Lot#21327) as the biotin labeling reagent, chooses 1:8 of 6B12 and 1:10 of 6H1as labeling condition, takes mixture of 6B12 and 6H1 as biotin-antibody (2 μg/ml as testing concentration); MPBS (pH 7.0, 0.1M) is the coating buffer solution; coating volume of anti-FGF23 is 100 μg; coating concentration is 2 μg/ml; sample diluent is casein solution; dilution ratio of streptomycin avidin is 1:3000; reaction time of enzyme and substrate is 10 minutes.

Implementation 2. Study of the detection threshold

According to the kit in implementation 1, conduct a three-steps dilution of FGF23 (10000 pg/ml and 2400 g/ml), evaluate and compare the lowest testing concentration and blank control and see whether p ≦0.05 or not. Results were seen in Table 14 and Table 15

TABLE 14
Study of the detection threshold of FGF23 reagent kit

| 1 | 2 | 3 | Average value | FGF23 (pg/m1) | T-TEST (p) |
|---|---|---|---|---|---|
| 2.269 | 1.998 | 1.938 | 2.068 | 10000 | ≦0.05 |
| 2.094 | 1.94 | 1.856 | 1.963 | 3333.3 | ≦0.05 |
| 1.598 | 1.045 | 1.007 | 1.217 | 1110.1 | ≦0.05 |
| 1.011 | 0.804 | 0.935 | 0.917 | 370.3 | ≦0.05 |
| 0.441 | 0.588 | 0.77 | 0.599 | 123.4 | ≦0.05 |
| 0.267 | 0.375 | 0.433 | 0.358 | 41.1 | ≦0.05 |
| 0.19 | 0.164 | 0.182 | 0.179 | 13.7 | ≦0.05 |
| 0.073 | 0.085 | 0.091 | 0.083 | 0 | — |

TABLE 15
Study of the detection threshold of FGF23 reagent kit

| I | 2 | 3 | Average value | FGF23 (pg/ml) | T-TEST |
|---|---|---|---|---|---|
| 1.739 | 1.637 | 1.717 | 1.698 | 12400 | ≦0.05 |
| 0.869 | 0.797 | 0.932 | 0.866 | 800 | ≦0.05 |
| 0.382 | 0.354 | 0.391 | 0.376 | 266.7 | ≦0.05 |
| 0.191 | 0.174 | 0.19 | 0.185 | 88.9 | ≦0.05 |
| 0.119 | 0.106 | 0.116 | 0.114 | 29.63 | ≦0.05 |
| 0.09 | 0.091 | 0.1 | 0.094 | 9.88 | ≦0.05 |
| 0.076 | 0.083 | 0.083 | 0.081 | 3.29 | ≧0.05 |
| 0.072 | 0.082 | 0.08 | 0.078 | 0 | — |

According to Table 14 and 15, the background level was the lowest when using casein as sample diluent. Data showed the lowest testing threshold limit value was 10 pg/ml.

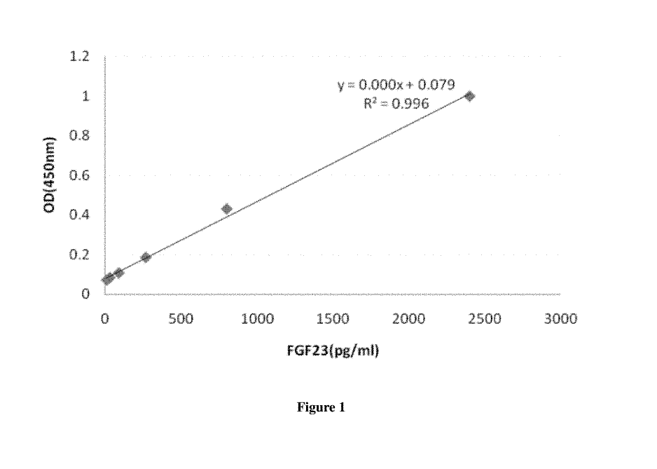

Use FGF23 at 2400 pg/ml, conduct three-times step dilution of it, then proceed coating according to the above optimal condition, practice color developing of TMB, and then study the linear relationship of absorbancy and sample concentration (see in FIG. 1). Experiment data revealed the linear range of FGF23 test kit was 2400 pg/ml˜10 pg/ml.

1. A chronic renal disease diagnostic kit, characterized by FGF23 monoclonal antibody, Sulfo-NHS-LC-Biotin, buffer solution, casein solution, streptomycin avidin and substrate TMB.

2. The diagnostic kit according to claim 1, wherein the said buffer solution is PBS (pH7.0, 0.1M).

3. The diagnostic kit according to claim 1, wherein the labeling condition is the weight ratio of FGF23 monoclonal antibody and Sulfo-NHS-LC-Biotin, which is 1:5˜1:12; the mixed concentration is 2 μg/ml.

4. The diagnostic kit according to claim 1, wherein the dilution ratio of the said streptomycin avidin is 1:3000, and the diluent is casein solution.

5. The diagnostic kit according to claim 1, wherein the action time of the said streptomycin avidin and substrate TMB is 10 minutes.

6. The diagnostic kit according to claim 1, wherein the threshold limit value of the said kit is 10 pg/ml, and the linear range of testing is 2400 pg/ml˜10 pg/ml.
